(19) United States
(12) Patent Application Publication
(10) Pub. No.: US 2014/0379268 A1
(43) Pub. Date: Dec. 25, 2014

(54) Method And Device For Preoperatively Predicting A Postoperative Horizontal Depth Of An Intraocular Lens In An Eye

(71) Applicants: Mario GERLACH, Hohen Neuendorf (DE); Eileen OSTBURG, Oranienburg (DE); Ronald BECKER, (US); Carl Zeiss Meditec AG, Jena (DE)

(72) Inventors: Ronald Becker, Potsdam (DE); Mario Gerlach, Hohen Neuendorf (DE); Stephanie Müller, Neuruppin (DE); Eileen Tschirpka, Oranienburg (DE)

(73) Assignee: CARL ZEISS MEDITEC AG, Jena (DE)

(21) Appl. No.: 14/114,875

(22) PCT Filed: May 3, 2012
(86) PCT No.: PCT/EP2012/058068

§ 371 Date: Feb. 6, 2014

(30) Foreign Application Priority Data
May 3, 2011 (DE) 10 2011 075 149.1

(51) Int. Cl. A61B 5/00; A61B 3/10; A61F 9/007

(52) U.S. Cl. CPC A61B 5/4851; A61F 9/007; A61B 3/10
USPC 702/19

(57) ABSTRACT

The present application relates to a method and a device for preoperatively predicting a postoperative horizontal depth of an intraocular lens in a patient's eye. Parameter values are provided, said parameters including a first parameter as the depth of an anterior chamber of human eyes, a second parameter as a horizontal length of human eyes, and a third parameter as a postoperative position of intraocular lenses in human eyes. Specific triplets of values are selected from said values as main interpolation nodes, each triplet of values comprising a value for each of the three aforementioned parameters, and the main interpolation nodes are entered into a three-dimensional coordinate system that is spanned by the three parameters. The main interpolation nodes are connected by edge connecting lines, and a predictive network that is unevenly formed in the three-dimensional coordinate system is generated at least using the positions of the edge connecting lines in order to predict the postoperative horizontal depth of the intraocular lens in the patient's eye.

### TECHNICAL FIELD

The invention relates to a method for preoperatively predicting a postoperative horizontal depth of an intraocular lens in a patient's eye, in which values of parameters are provided, wherein the parameters include a first parameter as a depth of an anterior chamber of human eyes, a second parameter as a horizontal length of human eyes and a third parameter as a postoperative position of intraocular lenses in human eyes. The invention also relates to a device for performing the method.

### PRIOR ART

It is known that in a human or animal eye, the natural lens can be removed, in particular by phacoemulsification, and can be replaced with an artificial intraocular lens (IOL). However, the inserted or implanted intraocular lens has to be very precisely attached location-specific in order to achieve the desired optical imaging characteristics and to be able to compensate for the visual defect of the eye to be corrected at best. Therein, intraocular lenses are known in very different configurations. Usually, they have an optical part and adjoining thereto one or more haptic parts. The intraocular lens is positionally retained in the eye with the haptic parts.

In order to be able to perform the correction of the visual defect at best by the intraocular lens, it is to be correspondingly configured positionally and shape-specific as well as with regard to the refractive power with regard to the visual defect to be corrected and the severity of the visual defect.

Therein, it is known that a corresponding intraocular lens is modeled by simulation and then manufactured and implanted into the eye.

Due to the plurality of different eyes with regard to the configuration and the dimensions, for example of the anterior chamber, the posterior chamber, the capsular bag, the thickness of the cornea, the curvature of the cornea, the horizontal length of the eye etc., an extremely high number of different situations can arise in connection with the respective visual defect of the specific eye and the severity of this visual defect, which make high demands to the modeling and manufacture of an intraocular lens.

For modeling such intraocular lenses with regard to the determination of the refractive power of the lens, different strategies and approaches are known. On the one hand, specific eye models are stored there, on the other hand, different assumptions and model calculations are taken as a basis.

For such predictions and modelings, it is essential to know the exact position of the intraocular lens in the eye to be treated in order to then be able to exactly determine the shape and/or the refractive power of the needed intraocular lens on the way of simulative modeling and then to be able to manufacture the needed intraocular lens based on this modeling. Therein, it is in particular then also essential that the predicted postoperative position of the intraocular lens in the eye for modeling the refractive power of the lens subsequently also coincides at best with the postoperative position in the eye after implantation. Only then, the visual defect to be corrected can be remedied at best with the intraocular lens.

The calculation of the refractive power of the lens can be effected based on different approaches and calculation algorithms. As already above mentioned, to this, a substantial influencing factor is the prediction and assumption of the postoperative position of the intraocular lens in the eye. A deviation of this predicted horizontal depth of the intraocular lens from the then actually present depth of the intraocular lens in the eye after surgery is a substantial main influencing factor for that if the result of surgery and the vision of the eye resulting from it can be evaluated as successful or not.

For determining an ELP (estimated lens position) as a postoperative predictive value, various approaches are known. For example, here, approaches according to the SRK method, approaches and modes of calculation according to Binkhorst, according to Shammas, according to Holladay or according to Haigis are mentioned. This enumeration is not conclusive and further model calculations are also known beyond that.

Therein, in some models, a three-dimensional coordinate system generated by the parameters of the preoperative depth of the anterior chamber of an eye, the horizontal length of an eye and a postoperative horizontal position of the intraocular lens in the eye is taken as a basis in simplified manner. In this three-dimensional coordinate system, for example in the approach of Haigis, then, a complete plane is generated. This plane disposed in the three-dimensional space then allows predicting the postoperative value of the position of the intraocular lens in the eye depending on the preoperative values of the chamber depth and the eye length. Due to the great simplifications and assumptions, these approaches provide too inaccurate predictions. On the one hand, they then effect the modeling for determining the refractive power of the lens, which in turn greatly adversely affects the actual corrective action of the modeled and manufactured intraocular lens in the natural eye. Thus, if first the predicted postoperative horizontal depth of the intraocular lens is made too inaccurately in modeling, this has substantial influence on the result of the vision after implantation.

From U.S. Pat. No. 5,282,852, a method for calculating the refractive power of an intraocular lens is known. Moreover, from US 2009/0251664 A1, a method as well as a system for determining the refractive power of an intraocular lens are also known.

Moreover, from DE 696 30 544 T2, a method for preoperatively selecting an intraocular lens to be implanted into an eye is known.

### PRESENTATION OF THE INVENTION

It is the object of the present invention to provide a method and a device, by which the prediction of a postoperative depth of an intraocular lens in the eye can be improved.

This object is solved by a method and a device according to the independent claims.

In a method according to the invention for preoperative prediction of a postoperative horizontal depth of an intraocular lens in a first eye to be treated or a patient's eye, in which the natural lens is to be replaced with an intraocular lens, values of parameters are provided, wherein the parameters include a first parameter as the depth of an anterior chamber of human eyes, a second parameter as the horizontal length of human eyes and a third parameter as the postoperative position of intraocular lenses in human eyes. From these values, specific value triplets with each one value of the three mentioned parameters are selected as main interpolation nodes, and the main interpolation nodes are entered into a three-dimensional coordinate system spanned by the three parameters. The main interpolation nodes are connected by edge connecting lines and a predictive network unevenly shaped in the three-dimensional coordinate system for predicting the postoperative horizontal depth of the intraocular lens in the patient's eye is generated at least by the positions of the edge connecting lines. The human eyes are not the patient's eye.

At least by the positions of the edge connecting lines, a predictive network unevenly shaped in the three-dimensional coordinate system and simulatively formed is generated, wherein a prediction of the postoperative depth of the intraocular lens in the first eye is effected based on this predictive network.

In particular depending on the measured parameters of the patient's eye, in particular the depth of the anterior chamber and the length of the patient's eye, and on the predictive network, the postoperative horizontal depth is determined and provided as information.

With the method according to the invention, thus, it is allowed that before the surgical procedure on the patient's eye for implanting an intraocular lens, it can be simulatively predicted where the intraocular lens will be disposed positionally with regard to the horizontal depth after implantation into the patient's eye. By the specific approach of generating main interpolation nodes and in particular the very specific connection of these interpolation nodes by edge connecting lines, an uneven predictive network is particularly advantageously generated. The precision of the prediction of this postoperative horizontal depth can be substantially improved with it. Since this postoperative horizontal depth is also an essential and crucial influencing parameter for the calculation and modeling of the intraocular lens with regard to the required refractive power, especially in cooperation with the more precise prediction of this postoperative horizontal depth, the modeling of the lens with regard to its shape and its refractive power can also be specified. In combination of these two precise modelable and predictable parameters, thus, best possible correction of the visual defect can also be achieved by the intraocular lens in the subsequent surgical procedure. Undesired deviations of the preoperative prediction of the postoperative horizontal depth and the then subsequent actual horizontal depth of the intraocular lens in the eye after the surgical procedure can thereby be avoided.

By the very specific configuration of the predictive network with the very specific shape with regard to an uneven design in the three-dimensional space, very different eye configurations and the very different requirements with regard to the determination of this depth associated therewith can be accommodated in improved manner. In particular, unusual or not current configurations of patient's eyes with regard to the mentioned parameters of the depth of the anterior chamber and the length of the eye can thereby be substantially better taken into account. In particular with eye shapes and dimensions occurring with less frequency in this respect, thus, the prediction of a postoperative horizontal depth can be substantially specified. Thereby, in the following, the required modeling of the intraocular lens with regard to its refractive power can also be improved and specified and thus the result of a surgical procedure can be substantially improved in particular for such rather rarely existing eyes.

In particular, the parameter of the horizontal depth of the intraocular lens in the patient's eye denotes the distance on the horizontal optical main axis of the eye between the cornea, in particular the front side of the cornea, and the vertical center plane or the vertical equatorial plane, below referred to as vertical equator, of the intraocular lens in the eye.

It can be provided that values of the other human eyes representing the database for generating and selecting the value triplets as main interpolation nodes can be measured. It can be provided that therein values of depths of anterior chambers and values of horizontal lengths of these other human eyes are measured in the other human eyes. In particular, then, after the surgical procedure on these other human eyes, the value of depths of implanted intraocular lenses is postoperatively measured. In particular, here, the value up to the front face of the lens is measured. Thereby, a very large and extensive database can be generated.

To this, the half of the thickness of the known inserted IOL is then in particular added to this value, in order to obtain a value comparative to the horizontal depth. Thereby, the accuracy is again increased.

Preferably, it is provided that values constituting the database for the main interpolation nodes are partially measured and partially estimated. In particular, it is provided that the values for the anterior chamber depth of the other human eyes constituting the database are measured. In particular, values for the length of other eyes constituting the database are measured. Preferably, values of postoperative depths of the intraocular lenses of human eyes constituting the database for the main interpolation nodes are estimated and/or calculated. In particular, these values can also be measured on the other human eyes.

It can also be provided that all of the values of the main interpolation nodes are estimated and/or calculated. The effort for one measurement is then omitted.

It is particularly advantageous if then estimated and/or measured and/or calculated values for the parameter of the anterior chamber depth are taken into account depending on which main interpolation nodes are to be formed. The accuracy of the predictive network is thereby increased.

In particular, by redundant value formation, in particular by distinct type of value formation, the accuracy can be increased and plausibility check can be effected.

Preferably, both estimated and measured values of the horizontal depth are taken into account for main interpolation nodes, which bound the predictive network at the edge.

Thus, the selection of main interpolation nodes can be effected in manifold and variable manner such that here too great flexibility is achieved. Thereby, the generation of the edge connecting lines and the resulting predictive network can also be configured and adapted in manifold manner.

By this variability and flexibility, the respective individual situations can be accommodated and a very dynamic approach can be achieved in establishing information for the preoperative prediction of this depth.

The predictive network is a definitely and individually generated construct as a statement framework for the depth of the intraocular lens to be determined. Therefore, it constitutes a presentable auxiliary tool, based on which the prediction of the depth is allowed in comprehensible and particularly precise manner. The framework shape is characterized by very definitely determined and formed interpolation nodes. Moreover, they are connected by defined geometric elements, namely straight lines. With regard to the statement precision and determination effort, this has particular advantages.

Preferably, it is provided that the postoperative depth of the intraocular lens in the patient's eye to be preoperatively predicted is determined depending on at least one connecting line contributing to generation of the predictive network. This is particularly advantageous since thereby the statement on the postoperative horizontal depth can be specified. Namely, to the effect that by the predictive network, a construct is already spanned, which particularly accommodates and correspondingly approaches the different eye dimensions and values by the uneven design, wherein by the contributing connecting lines, a particularly conclusive determination is then allowed. Namely, since the predictive network is virtually spanned by the connecting lines and these connecting lines thus virtually a priori constitute the main influencing factors for the calculation for the preoperative prediction of the depth.

Preferably, it is provided that the predictive network is generated from a plurality of partial meshes, which thus in particular represent partial networks.

Preferably, the main interpolation nodes are selected such that they each are vertices of at least one partial mesh. By such a configuration of the predictive network, the uneven design can be specified with regard to a more precise prediction of a postoperative horizontal depth of an intraocular lens depending on the predictive network. By the main interpolation nodes constituting the vertices of the partial meshes, the specification of the shape of the partial meshes, the size and the position in the three-dimensional coordinate system can be individually determined. Thereby, the prediction precision can be again increased since the partial mesh determination also in turn can be very variably and flexibly effected and can be determined very adapted to situation. By the interpolation nodes constituting the vertices of partial meshes, especially in context with the approach that these main interpolation nodes are connected by edge connecting lines, the approach in calculating the postoperative depth can be more exactly performed. The association of calculated values with specified partial meshes, which can then also be identified in position and/or size with regard to other partial meshes of the predictive network, is then possible in simpler manner and with less effort. Nevertheless, the statement precision is not impaired.

Preferably, it is provided that the partial meshes of the predictive network are spanned as rectangles. By all of the partial meshes having the same shaping and thus having four vertices, which are constituted by each four main interpolation nodes, a completely contiguous predictive network can be constructed. Therein, straight rectangles allow simply generating edge connecting lines and causing them to extend parallel with regard to a projection into a plane. The further calculation for a preoperative prediction of the depth of the intraocular lens is thereby possible in particularly simple manner and low in effort yet in extremely precise manner.

Preferably, it is provided that the partial meshes of the three-dimensionally forming flat predictive network are transferred to a projection plane or coordinate plane, wherein the coordinate plane is spanned by the mutually perpendicular coordinate axes for the parameters of the depth of anterior chambers and the length of eyes.

In particular, rasterization is then performed in the coordinate plane, wherein the partial meshes of the predictive network are taken as a basis for this rasterization as quadrants to this.

For predicting the depth, then, measured values of the patient's eye are then compared to the values of the quadrants with regard to the depth of the anterior chamber of the patient's eye and the length of the patient's eye, and from this it is determined, in which quadrant the patient's eye is classified. In particular, depending on the edge connecting lines of these quadrants, the depth of the intraocular lens in the eye to be predicted is then determined.

Preferably, it is provided that the rectangles are introduced into the coordinate system such that the edge connecting lines extend parallel to the coordinate axes between each two vertices in a partial mesh upon projection of the edge connecting lines into the coordinate plane, which is spanned by the mutually perpendicular coordinate axes for the parameters of the depth of anterior chambers and the length of eyes. This is to be particularly pointed out since thereby a very differentiated and conclusive predictive network can be formed. The covering of very much different combinations of pairs of values with respect to the parameters of the depth of the anterior chambers and the length of eyes can thereby be covered. Not least, by such a specific positional arrangement of the edge connecting lines, the calculation of the postoperative position of the intraocular lens and thus the value of the third parameter in this three-dimensional coordinate system can be greatly simplified and reduced in effort. Thereby, the determination of the preoperative prediction can also be very fast effected. This in particular has substantial advantages to the computational effort on the one hand and the information fast to be provided on the other hand.

Preferably, it is provided that the main interpolation nodes are selected such that the lengths of the edge connecting lines of the partial areas in the coordinate plane projected into the coordinate plane are identically generated in the direction of the first coordinate axis. Thereby, a very specific segmentation or quadrant formation can be effected.

Therein, it is preferably provided that the depth of the anterior chamber is plotted as the parameter on the first coordinate axis. Especially for this parameter, then, equally long partial meshes can be generated such that the actually measured values of eyes respectively coming within or encompassed can also be correspondingly grouped. By equaling the lengths with regard to this coordinate axis, statement on the frequency of such values in the respective quadrants can also be allowed with respect to the available number of already present data of the other human eyes.

Preferably, it is provided that the lengths of the edge connecting lines of the partial areas in the coordinate plane projected into the coordinate plane are differently generated in the direction of the first coordinate axis. Thereby, in particularly advantageous manner, a length dimensioning respectively adapted for the individual frequencies of the present values can be allowed. The statement precision can thereby be increased since especially if a plurality of values for a very specific quadrant range is present, a corresponding length configuration can be even more precisely effected.

Preferably, it is provided that at least three such partial lengths are formed along the first coordinate axis such that four main interpolation nodes are formed at least along this coordinate axis. Therein, it is preferably provided that the first main interpolation node is at a value of the depth of the anterior chamber of 1.50 mm. Preferably, a second main interpolation node with an identical value of the length of the eye as the first main interpolation node is at a value of 2.90 mm with respect to the depth of the anterior chamber. Preferably, it is provided that a third main interpolation node with an identical value of the length of the eye as the two first main interpolation nodes is at a value of 3.40 mm with respect to the depth of the anterior chamber. In particular, it is provided that a fourth main interpolation node with the same value of the length of the eye as the three first main interpolation nodes have, is at a value of 6.00 mm with respect to the depth of the anterior chamber.

Preferably, it is provided that the lengths of the edge connecting lines of at least two partial meshes in the coordinate plane projected into the coordinate plane are differently generated in the direction of the second coordinate axis. In particular, the length of the eye is plotted as the parameter on the second coordinate axis. With regard to the different length configuration, the analogous advantages result, as they were already above mentioned to the different length configuration of the partial meshes in the direction of the first coordinate axis.

Preferably, it is provided that along the second coordinate axis too, a plurality of further main interpolation nodes is plotted, which are all based on the same value of the parameter with respect to the depth of the anterior chamber. Thus, they vary only with regard to the value of the length of an eye. The statements made in this respect all relate to the situation if the main interpolation nodes and the edge connecting lines are projected into the coordinate plane, which is spanned by the two coordinate axes, on which the depth of the anterior chamber on the one hand and the length of the eye on the other hand are plotted.

Preferably, it is provided that the already above mentioned first main interpolation node is disposed at a value of 15.00 mm with respect to its value in the direction of the second coordinate axis. In particular, a further main interpolation node is disposed at a value of 22.50 mm with respect to the length of the eye. A further main interpolation node is in particular plotted at a value of 25.00 mm of the length of the eye and a further main interpolation node is plotted at a value of 40.00 mm of the length of the eye.

Preferably, it is provided that the length of an edge connecting line projected into the coordinate plane is generated in the direction of at least one coordinate axis depending on the number of the present preoperative values of the parameter plotted on this coordinate axis.

In particular, it is provided that this length is selected all the smaller, the greater the number of the preoperative values is. The prediction accuracy can thereby be improved.

Preferably, it is provided that the predictive network is matrix-like constructed and is generated with at least nine partial meshes. In particular, at least a 3×3 matrix is generated.

Preferably, depending on the pair of values of the depth of the anterior chamber and the length of the patient's eye, which values are measured, that partial mesh of the predictive network is selected, by which the pair of values is encompassed in the coordinate axes.

With regard to the determination of the values of the postoperative horizontal depths of the intraocular lenses in the other human eyes, on the one hand, measurements are taken into account. In addition to or instead of this, for these values of the main interpolation nodes in this third spatial direction, calculations or estimations of values can be taken into account. Thereby, a comparison and a more precise determination of these values can be effected such that the main interpolation nodes too are already very accurate and assume a very precise position. Thereby, the precision with respect to the position of the edge connecting lines is also improved, which in turn affects the precision of the uneven design of the predictive network in order to then be able to ensure a particularly precise preoperative prediction of a postoperative horizontal depth of the intraocular lens in the patient's eye, which constitutes the eye subsequently surgically to be treated, depending on this. By provision of the possibility of a predictive surface developing unevenly in the space based on the unevenly configured predictive network, very individually configured free shapes of this predictive network can result, but which positively benefits exactly the preoperative statement on the position of the intraocular lens in the patient's eye.

Preferably, it is provided that for the value of the depth of the anterior chamber of the patient's eye, a first intermediate connecting line contributing to the generation of the predictive network is determined depending on the values of the edge connecting lines of the selected partial mesh at the location of the value of the depth of the anterior chamber. In addition to or instead of this, it can also be provided that for the value of the length of the patient's eye, a second intermediate connecting line contributing to the generation of a predictive network is determined depending on the values of the edge connecting lines of the selected partial area at the location of the value of the length of the patient's eye. Thus, it is allowed that if specifically measured values of the patient's eye with respect to the depth of the anterior chamber and the length of the eye, which are then not explicitly on a main interpolation node, as they were selected, nevertheless furthermore a very precise preoperative prediction of the horizontal depth of the intraocular lens in the patient's eye can be effected by corresponding calculation.

In that the edge connecting lines can be described by exact linear equations and they each then extend parallel to the respective coordinate axes, in particular with projection into the coordinate plane spanned by the above mentioned parameters of the depth of the anterior chamber and the length of the eye, the calculation of the horizontal depth of the patient's eye can then also be effected very precisely. Namely, thus, after determination, in which quadrant or by which partial mesh the measured values of the patient's eye are captured, it can then also very specifically be resorted to the edge connecting lines framing this partial mesh, which were already previously determined by connecting the corresponding main interpolation nodes. If there the measured values of the depth of the anterior chamber of the patient's eye and the length of the patient's eye are then inserted in these edge connecting lines, thus, specific intermediate values for the depth of the intraocular lens can respectively be determined on the edge connecting lines. Since in particular with generation of a partial area in the form of a rectangle upon projection into the coordinate plane spanned by the parameters of the depth of the anterior chamber and the length of the eye, two opposing edge connecting lines are respectively present, upon specifically inserting the known measured values of the depth of the anterior chamber of the patient's eye and the length of the patient's eye, in turn, two intermediate interpolation nodes each appear on the respective edge connecting lines, which in turn can then be connected individually for generating the intermediate connecting lines.

Preferably, then, for all of the measured pairs of values of the depth of the anterior chamber of the patient's eye and the length of the patient's eye, corresponding values for a postoperative horizontal depth of an intraocular lens in the patient's eye appear, which can then be calculated from at least one intermediate connecting line.

Therein, it can be provided that the partial meshes between the edge connecting lines and to this the formed partial areas are flatly formed and are obliquely oriented in the space. With such a configuration, it is then sufficient that an intermediate connecting line is virtually generated only in one direction and thus only in one coordinate axis direction. Namely, since at the point of the pair of values of the depth of the anterior chamber of the patient's eye and the length of the patient's eye, the value of the horizontal depth can be calculated solely from this straight line and is equal to the calculated value, which results from a further intermediate connecting line generated in the second coordinate direction. Namely, since due to the flatly formed partial area, these two intermediate connecting lines exactly intersect at the point of the horizontal depth value to be predicted.

If the edge connecting lines are oriented such that a partial mesh with uneven partial area spans between them, thus, by the above explained approach, inaccuracy in the prediction can occur with only one intermediate connecting line. Since especially by the uneven configuration of the predictive network and also of the respective partial meshes, two intermediate connecting lines usually do not intersect with respect to the value of the horizontal depth of the intraocular lens in the patient's eye at the pair of values of measured depth of the anterior chamber of the patient's eye and measured length of the patient's eye, furthermore, a further decision scenario can be taken into account in this respect in order to improve the preoperative prediction.

Therein, it can in particular be provided that at the location of the pair of values of the depth of the anterior chamber and the length of the patient's eye, the calculated values of the postoperative depth of the intraocular lens in the patient's eye from the two intermediate connecting lines are compared, and the value of the postoperative depth of the intraocular lens in the patient's eye is determined depending on an occurring difference of the values. Therein, different approaches can be provided. Therein, the determination can be dependent on the magnitude of the difference on the one hand. However, in addition to or instead of this, it can also be provided that upon occurrence of such a difference, the further determination is effected depending on the quadrant or the partial mesh in the predictive network, with which the pair of values was associated or by which the pair of values was encompassed. This can be effected depending on the position of the partial mesh in the predictive network and/or depending on the size of the partial mesh compared to other partial meshes and/or compared to the predictive network.

In particular, it is provided that averaging of the differential value is performed for further determination, wherein it is in particular provided that the differential value is halved. This thus averaged value can then be in particular added to the smaller one of the value of the postoperatively predicted horizontal depth calculated by the intermediate lines.

Alternatively, it can also be provided that this averaged differential value is then subtracted from the greater one of the two values of the horizontal depth calculated by the intermediate connecting lines. This addition or subtraction of the averaged differential value too can in turn be effected depending on in which partial mesh the calculation is effected or by which partial mesh the measured pair of values of the patient's eye is encompassed.

It can be provided that the number of the main interpolation nodes and the edge connecting lines is selected as great as the predictive network constitutes a contiguous predictive surface, which unevenly extends in the space. In particular, based on the predictive network, such a predictive surface can also be formed by interpolation depending on the main interpolation nodes and the edge connecting lines. In addition, intermediate connecting lines for generating the predictive surface can also be taken into account.

In particular, the postoperative depth value determined according to the above explanation constitutes a predictive point on the predictive surface.

Thus, it can then also be provided that in progress of the prediction, such a great number of postoperatively determined depth values is present that the predictive surface is formed by these calculated depth values such that then subsequently the postoperative depth value can be preoperatively predicted for further eyes already from this unevenly curved predictive surface based on the measured pairs of values for the depth of the anterior chamber and the length of the eye, without additional further calculations having to be effected with intermediate and connecting lines.

Preferably, it is provided that for the determination of the postoperative depth value of the intraocular lens in the patient's eye for a provided intraocular lens with a haptic inclined with respect to the vertical and/or for a specific provided implantation location of the intraocular lens in the patient's eye, a first correction factor is taken into account. The calculation of the postoperative depth value then includes this first correction factor with the functional correlation.

In particular, a correction factor is taken into account, which is variably determined depending on the lens shape and/or the implantation location in the eye. Thereby, the prediction precision can be again increased. In particular for very specific combinations of required lenses for correcting corresponding visual defects in connection with different configurations of an eye to be surgically treated, then, a still more precise prediction of the postoperative position allows that the subsequent modeling is also very precisely allowed based on this preoperatively predicted depth value.

Preferably, it is provided that the first correction factor is determined by the following functional correlation:

Fakₖₒᵣᵣ=−a+[(Fak_{z}·b)−c],

wherein in particular a is between 1.3 and 1.43, b is between 0.45 and 0.65 and c is between 60 and 70, preferably between 64 and 66.

In particular, the following correlation applies:

Fakₖₒᵣᵣ=−1.39329+[(Fak_{z}·0.5663)−65.60]

Therein, Fak_{z} is a numerical value, which depends on the lens type and/or the location, at which the lens is to be implanted in the eye. It can also be a function of at least one lens parameter.

Moreover, it is preferably provided that the provided database is configured in compatible and consistent manner with respect to the values of the depth of an anterior chamber of the other eyes. Since there are different approaches in this respect, these values can have been measured based on different assumptions. Because it is possible in this context that this depth of the anterior chamber is measured up to the epithelial layer of the cornea or up to the endothelial layer of the cornea. Since the cornea has an average thickness of 0.50 mm, these obtained values are to be correspondingly matched such that the values of the other human eyes provided in the database are based on the same assumptions with respect to the measurement length.

For the further procedure of calculating the postoperative horizontal depth of an intraocular lens in the patient's eye, then, based on the measured values of the depth of the anterior eye chamber and the length of the patient's eye, the quadrant or the partial mesh of the predictive network is selected, in which these measured values of the patient's eyes with respect to these two parameters are encompassed.

In a further step, then, starting from the determined edge connecting lines of this specific partial mesh, which includes measured values of the patient's eye, the postoperative horizontal depth of the intraocular lens in the patient's eye is calculated and thus can then be preoperatively predicted.

Preferably, the predictive network and/or the determined and preoperatively predicted postoperative horizontal depth of the intraocular lens are displayed on a display unit of a device. In particular, the determined and preoperatively predicted postoperative horizontal depth of the intraocular lens is displayed on a display unit of a device as a value and/or in an eye illustrated as an image. To this, a symbolic sectional image or else a real image can be provided. The determined depth can also be displayed as overlay information in an image on the display unit.

Therein, these relations are true for an intraocular lens, the haptic of which is not inclined with respect to the vertical and which is to be implanted in the capsular bag of the eye. If an intraocular lens with inclined haptic is to be implanted in the first eye and/or the implantation is not to be implanted in the capsular bag, but in the anterior chamber or in the ciliary gap, thus, the calculation is to be performed considering the already above mentioned correction factor.

In particular, the Z factor Fak_{z}, on which the first correction factor depends, is determined with values between 110 and 125.

Therein, it is in particular provided that this Z factor Fak_{z} varies in value depending on the type of the intraocular lens. For example, the Z factor is 115.15 for an intraocular lens implanted in the anterior chamber. It is 115.60 for an iris-fixed intraocular lens. It is in particular 116.05 for a sulcus-fixed intraocular lens, which is plano-convex. It is in particular 116.90 for a sulcus-fixed intraocular lens, which is biconvex shaped. It is in particular 117.65 for an intraocular lens fixed in the capsular bag, which is plano-convex shaped. It is in particular 118.30 for an intraocular lens fixed in the capsular bag, which is biconvex formed.

It can also be provided that a once generated predictive network is corrected depending on subsequently obtained information about the actual positional depth of the intraocular lens in the patient's eye and in particular corresponding main interpolation nodes are changed. In particular, then, the predictive points on the predictive surface can also be correspondingly changed such that the uneven design can be adapted based on subsequently measured actual values of the eye.

In an advantageous implementation, it can be provided that a sorting is effected considering radii of the corneas of eyes. In particular, then, several matrices can also be created as predictive networks. In particular, this means that thus a predictive network is generated for a very specific radius of a cornea as it was set out above based on the procedures. Then, an own predictive network can in particular be established for each radius.

Moreover, further parameters, such as for example the thickness of the natural lens of an eye and/or the age of the patient and/or the ethnic origin can also be taken into account. As further biometric data, the boundary of the eyelids of an eye, the so-called WTW (white to white) ratio, can also be taken into account besides the corneal curvature.

In particular, for modeling an eye in calculating the refractive power for the intraocular lens, the Gullstrandt Model is taken as a basis. It is explicitly pointed out that the invention is not fixed to a specific eye model, but a preoperative prediction is possible independently of a specific eye model. As further eye models, for example, the Liou-Brennan model or the Holladay model can also be taken as a basis. They are only exemplary mentions for models, wherein the enumeration is not to be understood as conclusive.

The invention also relates to a method for modeling an eye lens, in particular an intraocular lens, in which the shape and/or the refractive power of the eye lens are determined and to this a postoperative horizontal depth of the eye lens in the patient's eye, in which the eye lens is to be implanted, is taken into account, wherein this depth is determined or predicted by an above explained method according to the invention or an advantageous implementation thereof. The eye lens to be implanted is then preoperatively selected based on the modeling.

The invention also relates to a device or a system for preoperative prediction of a postoperative horizontal depth of an intraocular lens in a patient's eye, which is formed for performing the method according to the invention or an advantageous configuration thereof. The system or the device includes at least one processor unit and a computer-readable storage medium to this, which is formed for communication with the processor.

In particular, it is provided that the system or the device has a memory, in which a plurality of values relating to the depths of the anterior chamber of other human eyes, values of the horizontal lengths of these other human eyes and values of postoperative depths of intraocular lenses in these other human eyes are stored. The memory can be a component of the system and the device, but can also be an external memory, which can be coupled to the system or the device.

Moreover, the system includes a plurality of instruction sequences provided for processing with the processor. These instructions are structured such that the processor is controlled to the effect that it selects and forms main interpolation nodes from values of specific value triplets of these above mentioned parameters. However, the main interpolation nodes are preferably preset in user-defined manner. Moreover, the instructions are adapted to the effect that these main interpolation nodes are entered into a three-dimensional coordinate system by the processor, wherein this coordinate system is spanned on the three coordinate axes by the parameters relating to the depth of an anterior chamber, the length of an eye and the postoperative horizontal depth of the intraocular lens.

Moreover, these instruction sequences are formed such that the processor is controlled to the effect that main interpolation nodes are connected by edge connecting lines. These edge connecting lines are calculated depending on the main interpolation nodes by the processor and the stored instruction sequences.

By the main interpolation nodes and the edge connecting lines, then, a predictive network unevenly shaped in the three-dimensional coordinate system and in particular simulatively formed is formed, which is generated for predicting the postoperative horizontal depth of the intraocular lens in the patient's eye.

In particular, the system is formed with further instruction sequences, which then determine, in particular calculate a postoperative horizontal depth of the intraocular lens in the patient's eye based on the entered measured values of the depth of the anterior chamber and the length of the patient's eye depending on the predictive network and in particular on one or more edge connecting lines and intermediate connecting lines.

To this, further instruction sequences are provided, by means of which the processor is controllable to the effect that it calculates intermediate connecting lines based on and depending on the edge connecting lines of a specific partial mesh or of a specific quadrant of the predictive network depending on these entered and measured values of the patient's eye and then determines, in particular calculates the postoperative horizontal depth value of the intraocular lens in the patient's eye preoperatively to be predicted depending on this.

Preferably, further instruction sequences are provided, which are formed for running the method steps of the method according to the invention or an advantageous configuration thereof.

Preferably, the system also includes stored tables on connecting lines and specific divisions of quadrants or partial meshes of the predictive network.

In particular, the system is also formed such that it is able to perform a determination, in particular calculation of the refractive power and/or of the lens shape depending on the preoperatively predicted postoperative horizontal depth of the intraocular lens in a patient's eye to be considered, in which the natural lens is removed and replaced with an intraocular lens.

In particular depending on this modeled calculation of the intraocular lens, it is then manufactured and furthermore then implanted in the patient's eye.

Preferably, it is provided that the above indicated formulas and correlations are stored in a memory of the system.

Particularly preferably, it is provided that the Z factor Fak_{z} is not preset as a specific static value, but that it is a function of a parameter of the intraocular lens, in particular of the parameter of the refractive power. Here, a linear function is preferably taken as a basis.

In particular, it is provided that with respect to the parameter of a postoperative horizontal depth of an intraocular lens, the horizontal path or the horizontal distance between the exterior of the cornea and the equator or the vertical center plane of the intraocular lens is denoted. Principally, however, another definition can also be taken as a basis, wherein then corresponding correction factors have to be taken into account with regard to compatibility and comparability of the values. This in particular relates to configurations, in which this parameter is not considered from the front side of the cornea, but from the rear side of the cornea. Similarly, this is to be correspondingly corrected if this parameter of the horizontal depth is measured to the effect that it is not characterized up to the equator of the lens, but only up to the front side of the lens.

Moreover, it is to be mentioned that the parameter of the depth of an anterior chamber can also be differently determined according to definition. Theoretically considered, the depth of the anterior chamber is defined from the rear surface of the cornea of the eye up to the front surface of the iris. Between these two interfaces, there is the anterior chamber. Between the rear surface of the iris and the vitreous body of the eye, there is the posterior chamber. Regarding measurement, the anterior chamber can be measured by means of ultrasound as well as by light, for example laser interferometry. Both methods slightly differ. On the one hand, in the physical kind of application as well as in the measurement result. The measurement of the distance from one interface to the next interface is identical to both methods. An interface is defined the more accurately, the greater the differences between the two media are with respect to light and sonic speed.

Thus, it can be measured from the front cornea surface or else from the rear cornea surface and thus from the front side or the rear side of the cornea. However, the difference of the boundary media is largest on the front side of the cornea. Accordingly, location of the interface is most exact on the front side of the cornea.

Thus, a preoperatively measured depth of the anterior chamber can be determined according to path by definition to the effect that it is measured from the front side of the cornea up to the front side of the natural eye lens. This is for example provided in the system IOL Master known from the applicant.

However, here too, another definition can be taken as a basis and the measurement can be effected from the rear surface of the cornea up to the front side of the natural lens. If the thickness of the cornea is then also measured, in turn, a compatible value comparison between the two possible definitions can be established. According to experience, a cornea thickness of 0.5 mm can be assumed as a very good average value.

Analogously, the postoperatively measured depth of the anterior chamber can be defined from the front side of the cornea up to the front side of the artificial eye lens and thus of the intraocular lens. However, here too, by definition, it can be measured from the rear side of the cornea up to the front side of the artificial eye lens.

With respect to the already above stated explanations, for the parameter of the postoperative horizontal depth of an intraocular lens in a first eye, which is to be preoperatively predicted, a distance on the horizontal main line of the eye is provided, which extends from the front side of the cornea up to the vertical center plane and thus to the equator of the intraocular lens. Then, this is in particular also the equator and thus the vertical center plane of the capsular bag. If an intraocular lens is provided, which has a non-angled haptic, thus, the haptic ends coincide with the equator of the intraocular lens. In particular if the intraocular lens also has a bisymmetrically distributed refractive power, the postoperative horizontal depth value of the intraocular lens in the first eye predicted by the method corresponds to the distance of the front cornea surface to the center of the intraocular lens.

As already also explained above, a first correction factor is to be provided if the haptic of the intraocular lens is angled and/or the intraocular lens is not to be implanted in the capsular bag. In particular, then, a correction factor is also to be used if the refractive power of the provided intraocular lens is asymmetrical on both curvature surfaces. In such configurations, the position of the vertical equator of the intraocular lens is to be adapted, which is effected with the already above explained Z factor Fak_{z}.

In particular, if in the approach for preoperative prediction of the postoperative horizontal depth of an intraocular lens in the eye, it is assumed that the vertical equator of the natural lens is always located at a certain location, and this position is independent of the type or model of an intraocular lens to be implanted, this location can also be calculated in advance, which in particular can be effected based on measured values of the eye length, the anterior chamber depth, the curvature of the cornea, the thickness of the natural lens and optionally also further parameters.

For the basic starting situation for predicting this horizontal depth, it is assumed that the lens has a non-angled haptic and is implanted in the capsular bag such that the vertical equator or the vertical equatorial plane of the intraocular lens then virtually coincides with the main plane, thus, the main plane of the intraocular lens is located in the same position as the equator of the capsular bag.

Corresponding corrections in the preoperative prediction to be calculated with regard to angled haptic and/or implantations at another location than the capsular bag and/or asymmetrically distributed refractive power of the front and the rear side of the intraocular lens, can be mapped with respect to the preoperative prediction of this depth by the mentioned correction factors.

Moreover, the invention also relates to a computer program product, on which instruction sequences for performing the method according to the invention or an advantageous configuration thereof are stored, in particular saved.

### BRIEF DESCRIPTION OF THE DRAWING(S)

Below, an embodiment of the invention is explained in more detail based on schematic drawings. There show:

FIG. 1 a vertical section through an eye by presenting specific parameters required for preoperative prediction;

FIG. 2 an enlarged vertical sectional illustration of an eye with an implanted intraocular lens and the presentation of the parameter of the postoperative horizontal depth postopeIOLE of the intraocular lens with non-angled haptics in the eye;

FIG. 3 a schematic illustration of a three-dimensional coordinate system with an exemplary predictive network;

FIG. 4 a projection illustration of the predictive network according to FIG. 3 in a plane, which is spanned by the coordinate axes, on which the parameters of the depth of the anterior chamber and the length of an eye are plotted;

FIG. 5 an exemplary illustration of a predictive network unevenly shaped in the space with the drawing of intermediate connecting lines;

FIG. 6 a further embodiment of a three-dimensional coordinate system with an unevenly shaped predictive network;

FIG. 7 an enlarged vertical sectional illustration of an eye with an implanted intraocular lens and the presentation of the parameter of the postoperative horizontal depth postopeIOLE* of the intraocular lens with angled haptics in the eye;

### PREFERRED IMPLEMENTATION OF THE INVENTION

In the figures, identical or functionally identical elements are provided with the same reference characters.

In FIG. 1, in a schematic vertical sectional illustration, a first eye or patient's eye 1 is shown, which has a visual defect to be corrected. In a surgical procedure, it is provided to remove a natural lens 2 disposed in a capsular bag 3. Then, an artificial eye lens in the form of an intraocular lens is to be inserted or implanted. The eye 1 has a cornea 4, which has a front side 5 and a rear side 6. Adjoining the cornea 4, an anterior chamber 7 is formed.

Moreover, the iris 8 of the eye 1 is drawn in.

The natural lens 2 also has a front side 9, which thus faces the cornea 4.

In the shown embodiment, it is provided that along a horizontal center line 10 and thus the optical main axis 10 of the eye 1, the parameter of the horizontal depth of the anterior chamber 7 is measured. This parameter is referred to as preopACD below.

It is measured in the embodiment on the optical axis 10 of the eye 1 from the front side 5 of the cornea 4 up to the front side 9 of the natural lens 2. It is measured by already above mentioned methods.

Moreover, a value of the parameter of the horizontal length of the eye 1 is also determined. This parameter is referred to as preopAL and also is measured along the optical axis 10. It extends from the front side 5 of the cornea 4 up to the retina 11 of the eye 1 in the embodiment. This value too is preoperatively measured for the patient's eye 1.

Moreover, in the illustration according to FIG. 1, a vertical center plane, also referred to as vertical equator 12 (equatorial plane), of the capsular bag 3 is represented.

In order to now correct the visual defect of this patient's eye 1 at best, it is required to be able to preoperatively predict and model at best the position and the configuration of the intraocular lens to be implanted.

For preoperative prediction of a postoperative horizontal depth of an intraocular lens to be implanted in this eye 1, below, the corresponding approach for prediction is explained in more detail.

To this, first, a device or a system is provided, which has a computing unit with at least a processor and a storage unit. By means of an input unit, the measured values of the parameters preopACD and preopAL of the eye 1 can be input.

In the device, a plurality of instruction sequences is stored, which can for example be stored on a computer program product in the form of a storage medium. In the device, communication between the processor and the storage medium exists such that it is correspondingly controllable, which instruction sequences the processor is to select and run and on which basis of the input and stored values calculation for preoperative prediction of a postoperative horizontal depth of an intraocular lens in the eye 1 is to be effected.

In the device, in a data storage, a plurality of values are stored, which include values of the parameter preopACD and thus of the depth of the anterior chambers of other human eyes and values of the parameter preopAL and thus of horizontal lengths of these other human eyes. Moreover, in this data storage, postoperatively measured values of a third parameter, namely the postoperative depth of intraocular lenses in these other human eyes, are also stored. However, estimated values of the postoperative horizontal depth of intraocular lenses are in particular provided. The values of the other human eyes, which are used as a database for the further procedure in the preoperative prediction of the postoperative horizontal depth of an intraocular lens in the patient's eye 1, are thus collected and stored and in particular also continuously updated.

It can also be provided that values, in particular all, are estimated and/or calculated.

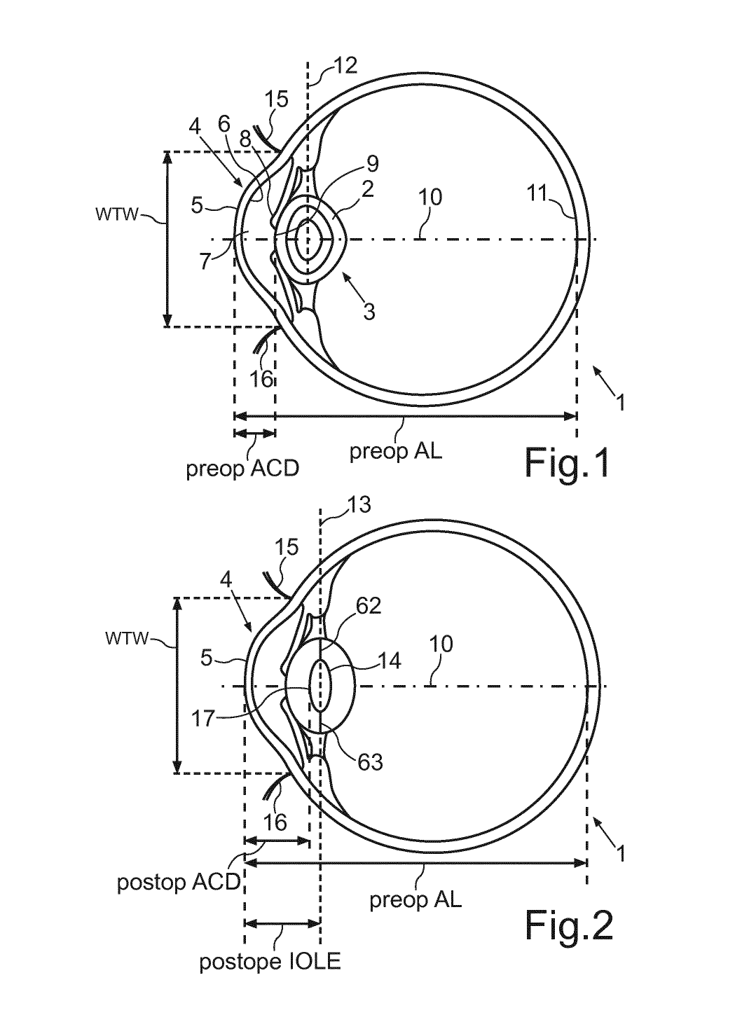

With regard to the parameter of the horizontal depth of an intraocular lens referred to as postopeIOLE according to the illustration in FIG. 2, it is measured in the embodiment from the front side 5 of the cornea 4 up to a vertical center plane 13, also referred to as a vertical equator or main plane, of the intraocular lens 14.

According to the illustration in FIG. 2, the exemplary illustration of the eye 1 is shown such that it is also in particular shown simulatively modeled.

In particular, in the preoperative predictive determination of the depth value and thus of the value of the parameter postopeIOLE, consideration of further biometric parameters of the eye 1 can be effected. Herein, the curvature of the cornea 4, the thickness of the natural lens 2 along the optical axis 10 can be measured as biometric parameters and the WTW ratio can be taken into account. Therein, the WTW ratio is measured in vertical direction between the contact points of the eyelids 15 and 16 on the eye 1.

For further explanation of the procedure for preoperative prediction of the value for the parameter postopeIOLE for the embodiment in FIG. 2, then, based on the provided database of a plurality of other human eyes, a selection of main interpolation nodes is performed. Therein, based on the available values for the three mentioned parameters preopACD and preopAL as well as values of horizontal depths of an intraocular lens postoperatively measured for the other eyes, value triplets are formed.

In particular, the main interpolation nodes are formed based on estimated values of the parameters and/or of measured values of these parameters on other human eyes than the patient's eye, in particular formed in user-selected manner. When estimated and/or when measured values are taken into account, depends on the main interpolation node respectively to be generated, in particular the position thereof from the other main interpolation nodes. In particular, the values of the anterior chamber depth and the values of the eye length of the other eyes are only measured.

As also exemplarily drawn in the illustration according to FIG. 2, this postoperatively measured value of the intermediate position referred to as postopeACD as the parameter, is measured between the front side 5 of the cornea 4 and the front side 17 of the intraocular lens 14.

It is to be mentioned that the illustration in FIG. 2 shows an already implanted IOL in order to be able to present and explain the individual parameters. Naturally, the IOL is not yet implanted in the eye 1 when the method according to the invention for preoperative prediction of the postoperative horizontal depth is performed.

With regard to the provided and stored database of these value triplets of the other human eyes, first, it is also to be matched if these obtained values are also compatible and are based on the same assumption of the path length definitions. As already above mentioned, in this respect, different definitions can be at the basis, and the compatibility is to be established to the effect that optionally the thickness of the cornea 4, in particular along the optical axis 10, is also taken into account.

Thus, the database is then to be provided to the effect that the values, in particular all of the values are provided on the same definition basis for the parameters preopACD, preopAL and postopeIOLE.

As already mentioned, a plurality of main interpolation nodes is then formed from specific value triplets, which are then drawn into a three-dimensional coordinate system.

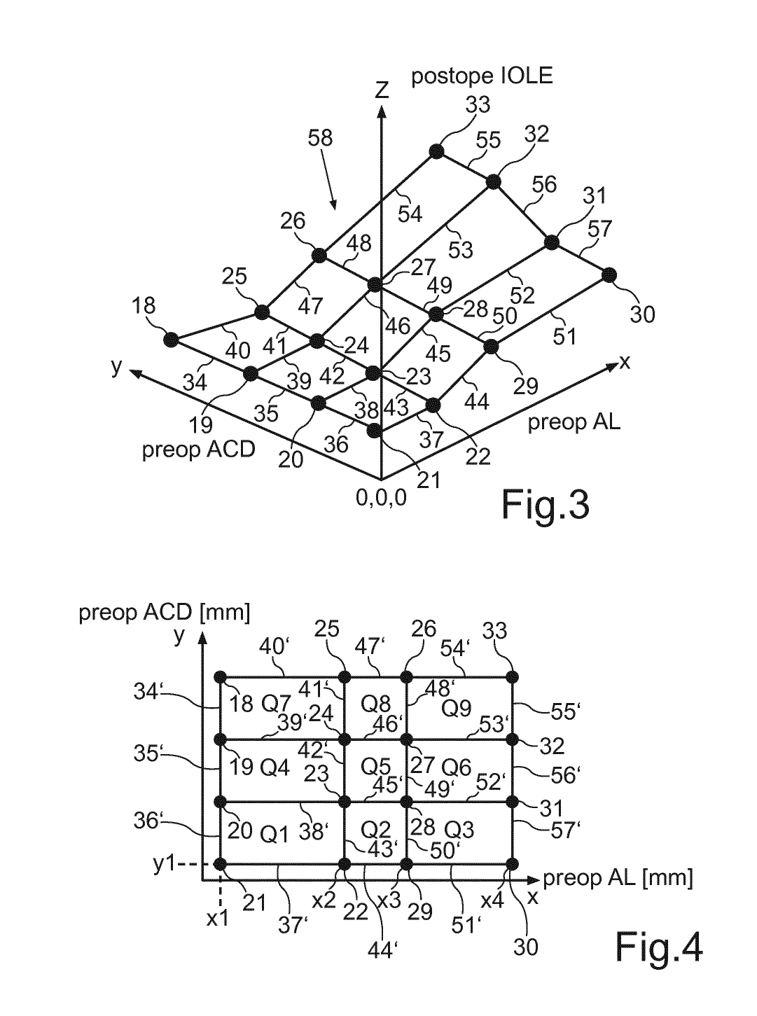

This is illustrated in FIG. 3, wherein therein the parameter preopAL is plotted on the x-axis, the parameter preopACD is plotted on the y-axis and the parameter postopeIOLE is plotted on the z-axis. The coordinate axes are perpendicular to each other.

From the selected value triplets, then, the already mentioned interpolation nodes are drawn into this three-dimensional coordinate system. In the embodiment, it is provided that 16 main interpolation nodes 18 to 33 are drawn in.

These main interpolation nodes 18 to 33 are selected in the embodiment such that they each constitute vertices of rectangular partial areas if they are projected into the x-y plane. This is shown in the illustration according to FIG. 4.

The main interpolation nodes 18 to 33 are then connected by edge connecting lines. These edge connecting lines are calculated, wherein therein the adjacent main interpolation nodes are respectively taken into account such that an edge connecting line is calculated from two adjacent main interpolation nodes due to a linear equation. Thus, edge connecting lines 34 to 57 are formed. Thereby, the predictive network 58 unevenly shaped in the space drawn in FIG. 3 arises.

From the following table 1, exemplary values for the main interpolation nodes 18 to 33 can be taken. Similarly, the linear equations for the edge connecting lines are entered there in a coordinate direction.

TABLE 1

| | | | | | | | |
|---|---|---|---|---|---|---|---|
| preopACD | preopAL | 15.00 | 22.50 | 22.50 | 25.00 | 25.00 | 40.00 |
| | Z | 2.250 | 3.100 | 3.100 | 3.500 | 3.500 | 4.50 |

| | | | | |
|---|---|---|---|---|
| 1.50 | 2.250 | Z_{RV36}^{x1} = 0.9842857 • ACD + | Z_{RV43}^{x2} = 0.7142857 • ACD + | Z_{RV50}^{x3} = 0.75 • ACD + 2.375 |
| | | 0.8035714 | 2.0285714 | |
| 2.90 | 3.600 | Z_{RV43}^{x2} = 0.7142857 • ACD + | Z_{RV50}^{x3} = 0.75 • ACD + 2.375 | Z_{RV57}^{x4} = 0.2857143 • ACD + |
| | | 2.0285714 | | 4.0714288 |
| 2.90 | 3.600 | Z_{RV35}^{x1} = 0.5 • ACD + 2.15 | Z_{RV42}^{x2} = 0.49988 • ACD + | Z_{RV49}^{x3} = 0.0 • ACD + 4.55 |
| | | | 2.85348 | |
| 3.40 | 3.850 | Z_{RV42}^{x2} = 0.49988 • ACD + | Z_{RV49}^{x3} = 0.0 • ACD + 4.55 | Z_{RV56}^{x4} = 0.7 • ACD + 2.87 |
| | | 2.650348 | | |
| 3.40 | 3.850 | Z_{RV34}^{x1} = 0.8269231 • ACD + | Z_{RV41}^{x2} = 0.6346385 • ACD + | Z_{RV48}^{x3} = 0.5576923 • ACD + |
| | | 1.0384615 | 2.1921692 | 2.6538462 |
| 6.00 | 6.000 | Z_{RV41}^{x2} = 0.6346385 • ACD + | Z_{RV48}^{x3} = 0.5576923 • ACD + | Z_{RV55}^{x4} = 0.7884615 • ACD + |
| | | 2.1921692 | 2.6538462 | 2.5692308 |

Therein, Z denotes the function of the equation for the edge connecting line (RV) at the location xi. Therein, Z is a function of y, wherein y is the parameter preopACD. For clarity, Z is indicated without this variable and only ACD is described in the equation instead of preopACD. Exemplarily, only the equations for straight lines are indicted, which extend parallel to the y-axis in FIG. 4. Similarly, the equations for the straight lines extending parallel to the x-axis in FIG. 4 could also be indicated.

As is apparent, the predictive network 58 is spanned from a plurality of partial areas or partial networks, wherein a partial network of at least one partial mesh is respectively bounded by four main interpolation nodes 18 to 33 disposed adjacent to each other and the edge connecting lines 34 to 57 respectively connecting these four main interpolation nodes 18 to 33 to each other. As is apparent, the predictive network 58 is spanned from the plurality of partial areas or partial networks directly contiguous to each other and adjoining each other. If the partial networks are flatly considered, thus, they have different slopes and curvatures in the three-dimensional coordinate system.

To the embodiment, at least some areas of partial meshes are unevenly formed in the space, which arises by the position of the associated main interpolation nodes.

As is apparent from the illustration in FIG. 4, the main interpolation nodes 18 to 33 are selected such that they span a rectangle upon projection into the x-y plane. To this, it is provided that a plurality of interpolation nodes, in the embodiment the interpolation nodes 18 to 21, have different preopACD values, but an identical preopAL value. The same applies to the main interpolation nodes 22 to 25. These main interpolation nodes 22 to 25 are selected at a different preopAL value, but have the same preopACD values as the main interpolation nodes 18 to 21. Analogously, this also applies to the main interpolation nodes 26 to 29 and 30 to 33, which also each have the same preopACD values, but are disposed at different preopAL values.

As is moreover apparent from the illustration in FIG. 4, for example, by the main interpolation nodes 20, 21, 22 and 23, a first quadrant Q1 is rectangularly spanned, wherein the main interpolation nodes 20 to 23 constitute the vertices of this rectangle. Again, this is to be considered with regard to the projection into the x-y plane (FIG. 4).

The main interpolation nodes 18 to 33 are selected such that the sizes of the x-y-projected partial networks of the predictive network 58 in the form of the rectangular quadrants Q1 to Q9 are adapted in their area size, namely to the number of values from the database of the other human eyes respectively present in these pairs of values.

In particular, it is provided that these rectangular quadrants Q1 and Q9 projected into the x-y plane in FIG. 4 are configured the smaller, the greater the number of values of the underlying database in these area regions is. Thereby, the accuracy in the further predictive determination of the postoperative horizontal depth value and thus of a value of the parameter postopeIOLE is increased.

As is apparent, in the embodiment, it is provided that the predictive network 58 is spanned from nine partial networks or partial meshes and thus in particular a 3×3 matrix is generated.

The number of the partial networks and thus in particular also that of the x-y-projected flat rectangular areas with the quadrants Q1 to Q9 is therefore only exemplarily. Another number of such partial networks can also be provided and the configuration as a 3×3 matrix is also only exemplary.

Preferably, it is provided that the main interpolation nodes 18 to 21 are at a value of preopAL=15.00 mm. In particular, in the embodiment, it is provided that the main interpolation nodes 22 to 25 are at a value of preopAL=22.50 mm. In particular, the main interpolation nodes 26 to 29 are at a value of preopAL=25.00 mm. Furthermore, the main interpolation nodes 30 to 33 are at a value of preopAL=40.00 mm.

In the embodiment, moreover, it is provided that the main interpolation nodes 21, 22, 29 and 30 are at a value of preopACD=1.50 mm. Furthermore, it is in particular provided that the main interpolation nodes 20, 23, 28 and 31 are at a value of preopACD=2.90 mm. Moreover, it is provided that the main interpolation nodes 19, 24, 27 and 32 are at a value of preopACD=3.40 mm. Moreover, it is in particular provided that the main interpolation nodes 18, 25, 26 and 33 are at a value of preopACD=6.00 mm.

From these exemplary values, then, the sizes of the projected rectangles with respect to the quadrants Q1 to Q9 can also be calculated and in particular the size ratios relative to each other between the quadrants Q1 to Q9 can also be determined.

Since for each of the main interpolation nodes 18 to 33, the z-values and thus the values of the parameter postopeIOLE are also known, here, a linear equation can be established for each of the edge connecting lines 34 to 57.

Herein, in table 1, the above mentioned values of the main interpolation nodes 18 to 33 for the parameters preopACD, preopAL and as the z value the values of the parameter postopeIOLE are exemplarily entered. Based on this, the linear straight line equations for the edge connecting lines 34 to 57 represented in table 1 can be calculated. In the table 1, for clarity, only the linear equations for the edge connecting lines 34 to 36, 41 to 43, 48 to 50 and 55 to 57 are represented.

With regard to the nomenclature, for example, in equation Z_{RY36}^{x1} for the edge connecting line 36, the superscript x1 means that the straight line was determined for the constant value or at the location x1 of the parameter preopAL, wherein this is at the value of x1=15 at the two main interpolation nodes 20 and 21, between which the equation for the edge connecting line 36 extends. Herein, the notation RV36 subscript in the function z relates to the edge connecting line 36. It extends between the main interpolation nodes 20 and 21 such that a nomenclature HS 21-20 could also be used.

Corresponding construction and interpretation is then also to be understood for the other linear equations indicated in the table. For example, since the linear equation with the superscript x2 and the subscript RV 43 is an equation for an edge connecting line 43 associated with the quadrant Q1 and associated with the adjacent and adjoining quadrant Q2, these equations are identical in table 1.

The predictive network 58 can be configured arbitrarily more fine meshed depending on the number of the selected main interpolation nodes and the partial mesh number resulting from it.

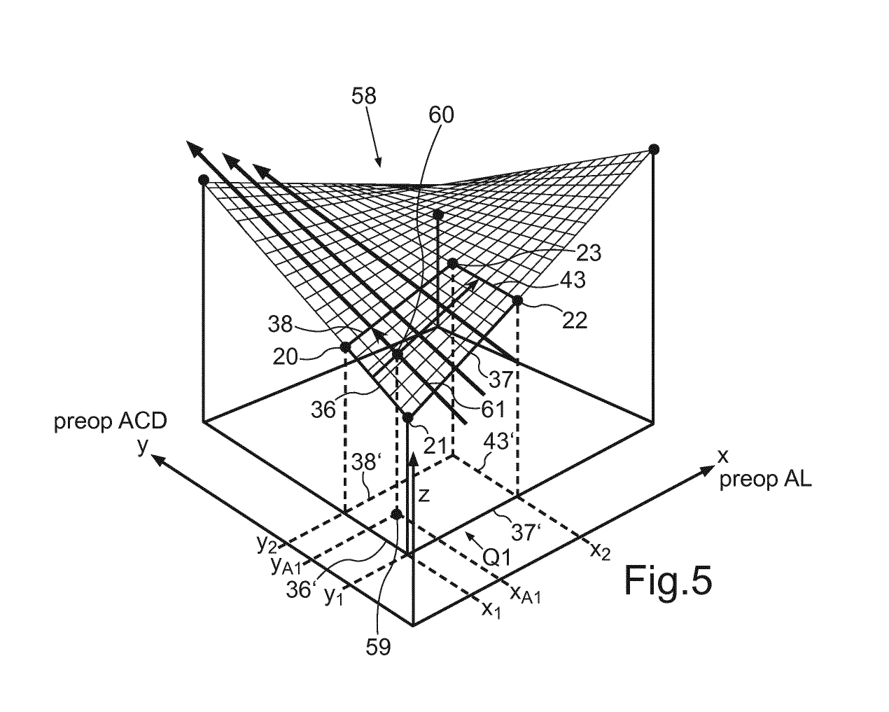

Based on the spanned predictive network 58, then, furthermore, according to the illustration in FIG. 5, a calculation for preoperative prediction of a depth value for the parameters postopeIOLE for the first eye 1 is performed.

The illustration of the predictive network 58 shown in FIG. 3 can be generated independently of the first eye. Rather, it is in particular generated exclusively on measured and/or calculated and/or estimated values for the plurality of different other eyes and the thereby underlying database.

The areas of the partial meshes of the predictive network 58 formed between the edge connecting lines are differently inclined and oriented partial areas in the space due to the different orientations of the respectively underlying edge connecting lines, which represent the respective boundaries of the partial networks in the shown embodiment. This can in particular be understood to the effect that the respective partial networks and in particular the edge connecting lines are connected by correspondingly spanned partial areas. Due to the different inclinations of the edge connecting lines, however, a partial area of a partial network is usually not a plane, but also an area again specifically curved in the three-dimensional space.

Based on the predictive network explained according to FIG. 3 and FIG. 4, which is the predictive network 58 in the embodiment, furthermore, for the specific eye 1 to be examined, a preoperative prediction for the depth value and thus a value of the parameter postopeIOLE can then be effected.

Therein, according to the illustration in FIG. 5, first, the pair of values including the preoperatively measured value preopACD representing the value yA1 in the illustration according to FIG. 5, and the preoperatively measured value preopAL of the eye 1, by which the value xA1 is characterized, is taken as a basis. Depending on these two values yA1 and xA1, then, that quadrant Q1 to Q9 is selected, by which the pair of values of these parameters of the eye 1 is encompassed. Exemplarily, in the embodiment, it is provided that the pair of values yA1 and xA1 is encompassed by the quadrant Q1, and thus a further calculation based on the partial mesh bounded by the main interpolation nodes 20, 21, 22 and 23 at the vertices is performed in the predictive network 58. This partial mesh is bounded by the edge connecting lines 36, 37, 38 and 43.

Based on this point 59 in the x-y plane, which is characterized by the pair of values yA1 and xA1, then, intermediate connecting lines are further generated.

They are again given as linear equations.

To this, according to the following formulas 1 and 2, for which the generalizations of the formulas exemplarily specified in the previous table 1 for specific values and embodiments are mentioned, the calculation is performed.

Z_{RV36}^{x1}(y)=m_{20|20}y+n_{21|20} (1)

Therein, the linear equation for the edge connecting line 36 results from the formula 1 and the linear equation for the edge connecting line 43 results from the formula 2.

In these formulas, the factors m with their subscripts each denote the slope between the two main interpolation nodes 20 and 21 on the one hand and the main interpolation nodes 22 and 23 on the other hand. Therein, y denotes the respective value along the y-axis and thus characterizes a value for the parameter preopACD. Therein, the factors n with their subscripts respectively characterize, as it is usual in mathematic linear equations, the z-value if y=0 is substituted for this value.

Correspondingly, the formulas 3 and 4 represent the general linear equations for the edge connecting lines 37 and 38.

Z_{RV37}^{y1}(x)=m_{21|22}x+n_{21|22} (3)

Z_{RV38}^{y2}(x)=m_{20|23}x+n_{20|23} (4)

Therein, the equation according to formula 3 is determined at the constant y-value y1 and the linear equation according to formula 4 is determined at the constant y-value y2. Therein, y1 represents that y-value of the main interpolation nodes 21 and 22, whereas y2 represents the y-value of the main interpolation nodes 20 and 23.

Based on these four formulas, then, a first intermediate connecting line (ZV) 60 is determined. This is effected according to the following formula 5.

$${Z}_{ZV 50}^{yA 1} ( x ) = \frac{{Z}_{RV 43}^{x 2} ( yA 1 ) - {Z}_{RV 36}^{x 1} ( yA 1 )}{{x}_{2} - {x}_{1}} \cdot x + {n}_{ZV 60} \qquad ( 5 )$$

With respect to the slope of this straight line, therein, a difference between the values of the formulas 1 and 2 at the location yA1 is determined, and this value is divided by the difference of the values x2 and x1 for determining the slope. Therein, n again denotes the z-value if x=0 is substituted for the value in the formula 5.

The formula 5 then thus determined then represents the equation for the first intermediate connecting line 60. Upon projection of this intermediate connecting line 60 into the x-y plane, it represents a parallel to the edge connecting lines 37′ and 38′ correspondingly projected into the x-y plane.

If then the measured value xA1 of the eye 1 is then substituted into this equation according to formula 5, thus, one obtains the z-value of this eye 1, which then also represents postopeIOLE of the intraocular lens in the eye 1 as a preoperative predictive value for a postoperative horizontal depth.

For improving the preoperative prediction, in particular, it can be additionally provided that a second intermediate connecting line 61 is determined. It is calculated according to the following formula 6, wherein here again the slope, which is then multiplied by y, is calculated analogously to the formula 5, wherein here the difference of the values of the formulas 3 and 4 is calculated at the value xA1 and divided by the difference of the values y2−y1.

$${Z}_{ZV 61}^{xA 1} ( y ) = \frac{{Z}_{RV 38}^{y 2} ( xA 1 ) - {Z}_{RV 37}^{y 1} ( xA 1 )}{{y}_{2} - {y}_{1}} \cdot y + {n}_{ZV 61} \qquad ( 6 )$$

Based on formula 6, there, by substituting the value yA1, the z-value of the second intermediate connecting line 61 can then be determined.

This is advantageous to the effect since due to the shaping of the three-dimensional partial mesh of the quadrant Q1, which is bounded by the edge connecting lines 36, 37, 38 and 43, the two intermediate connecting lines 60 and 61 do not intersect at the location xA1 and yA1, which means that the z-values resulting from it are different.

In order to now obtain a best possible and precise predictive result for the value of the parameter postopeIOLE, then, depending on these two z-values of the intermediate connecting lines 60 and 61, it can be further differently proceeded. Thus, it can for example be provided that the difference of these two z-values is averaged, wherein in particular these values are then subtracted from each other and the amount of this difference is divided by two. In particular, it can then be provided that the averaged differential value is added to the smaller one of the two z-values resulting from intermediate connecting lines. However, similarly, it can also be provided that this averaged differential value is subtracted from the greater one of the two z-values resulting from the intermediate connecting lines.

The approach, how it is proceeded, if two different z-values result from the intermediate connecting lines in order to then make a statement as precise as possible for the preoperative prediction of the postoperative horizontal depth of the intraocular lens in the eye, can then depend on a variety of circumstances and parameters. Thus, for example, this can be effected depending on the magnitude of the differential value and/or be dependent on the specific quadrants Q1 to Q9, in which the pair of values xA1, yA1 is located, and/or dependent on the size and/or position of the quadrant Q1 to Q9, in which this pair of values is located. Of course, the statements with respect to the quadrant apply in analogous manner also to the partial areas or partial networks of the predictive network 58 located in the three-dimensional space, which cover the pair of values xA1, yA1.

In this respect, then, this predicted z-value and thus the value of the postoperative horizontal depth postopeIOLE of the intraocular lens in this first eye 1 is calculated. It is then taken into account as a further value, which is essential, in the calculation of the refractive power of the intraocular lens to be implanted in the first eye 1.

The just mentioned explanations are based on the specification that the intraocular lens 14 has haptics 62 and 63 according to the illustration in FIG. 2, which are not inclined forwards or backwards with respect to the vertical. Moreover, it is provided that the intraocular lens 14 is to be implanted in the capsular bag 3 of the eye 1.

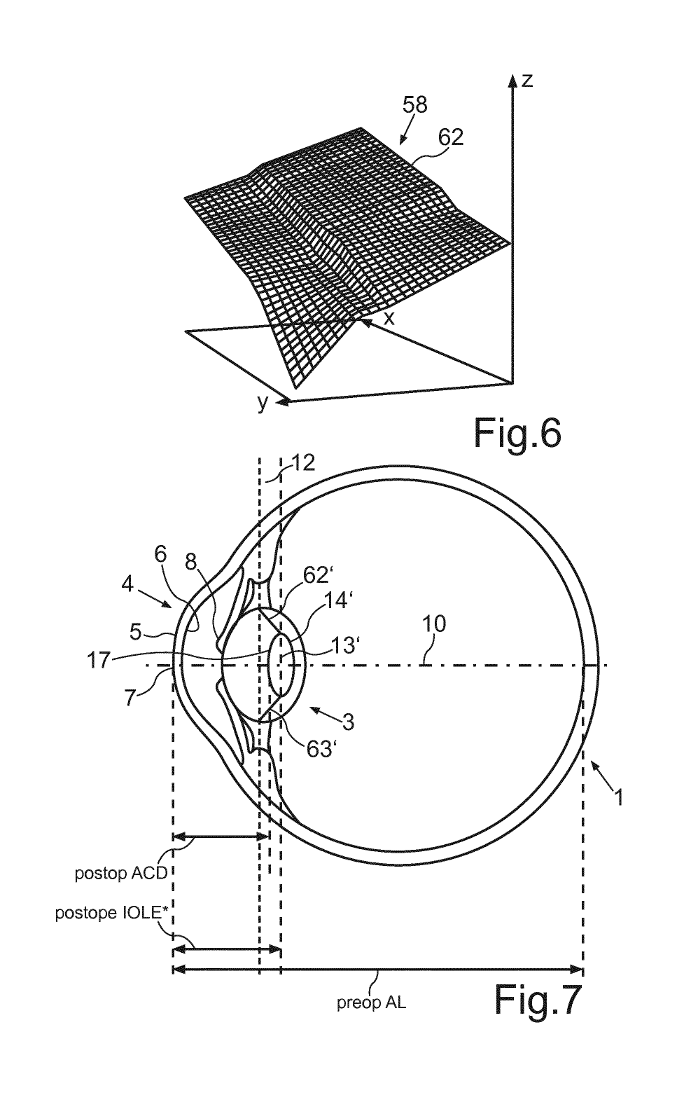

In FIG. 6, an exemplary illustration of an unevenly formed predictive network 58 is shown, which is disposed in the three-dimensional coordinate system. The different inclinations and curvatures can be recognized. It can also be provided that the predictive network 58 constitutes a completely contiguous predictive surface for example by interpolation based on the provided edge connecting lines. It can then be configured according to the predictive surface 62 in FIG. 6.

If such a contiguous predictive surface 62 is generated, thus, it can also be provided that depending on the measured values xA1 and yA1 further calculations of intermediate connecting lines and optionally of z-values of the intermediate connecting lines to be determined do not have to be performed, but that explicitly from the present predictive surface 62, then already a single z-value can be withdrawn. In this respect, the computational effort for determining the z-value is lower after the predictive network 58 and consequently the predictive surface 62 were already determined. However, compared to the approach as it was set out to FIG. 3 to FIG. 5, a greater preceding computational effort is required to be able to determine and provide the predictive surface 62 in correspondingly accurate and precise manner at all. Therein, it is in particular provided that a very high number of main interpolation nodes is provided to be able to generate a particularly close-meshed predictive network 58, which then allows a corresponding exactly and unevenly shaped predictive surface 62 for example by interpolation.

Based on the explanations to the specific configuration in FIG. 2, then, the thus simulatively determined predicted depth and thus the preoperatively predicted postoperative horizontal depth value of the intraocular lens in the eye 1 is modeled, and the refractive power and the shape of the intraocular lens 14 is then modeled from it. The intraocular lens 14 thus manufactured then based on these modeling values is then correspondingly implanted into the eye 1. Then, by measuring the parameter value of the parameter postopACD drawn into FIG. 2, the predicted position of the intraocular lens 14 can be checked. In FIG. 2, the then already implanted state of the intraocular lens 14 is exemplarily drawn in the simulation model of the eye 1.

In a further implementation, it can be provided that the intraocular lens 14′ is forward angled with respect to the vertical and thus has inclined haptics 62′ and 63′ as it is shown in FIG. 7. In order to also be able to allow correspondingly precise predictions for the postoperative horizontal depth value postopeIOLE* for such constellations, the above mentioned and set out formulas 5 and 6 are to be modified. In particular, therein, a formula 7 and optionally 8, as they are presented below, are then to be used.

Z_{ZV60}^{yA1}(x)*=Z_{ZV60}^{yA1}(x)−1.39329+[(Fak_{Z}·0.5663)−65.60] (7)

Z_{ZV61}^{xA1}(y)*=Z_{ZV61}^{xA1}(y)−1.39329+[(Fak_{Z}·0.5663)−65.60] (8)

The different circumstances with an angled haptic of the intraocular lens and/or an implantation with the capsular bag of the eye 1 are taken into account in it.

The Z-factor Fak_{Z} can be a static preset value varying depending on the lens type and the lens shape IOL as well as the attachment at specific location in the eye. In particular, this value can vary between 110 and 125.

It is particularly advantageous if this factor Fak_{Z} itself in turn is a function, which in particular depends on a specific parameter of the provided intraocular lens, in particular is dependent on the refractive power. Here, in particular a linear function could preferably be taken as a basis.

With respect to the explained approach to FIG. 5, first, the intermediate connecting line 61 can also be determined and be sufficient alone for the z-value determination and thus the value postopeIOLE. Optionally, subsequently, the intermediate connecting line 60 can then be determined and the further procedure can be effected as explained in FIG. 5. The same applies to the formulas 7 and 8.

1. Method for preoperative prediction of a postoperative horizontal depth (postopeIOLE, postopeIOLE*) of an intraocular lens in a patient's eye, in which values of parameters are provided, wherein the parameters include a first parameter as the depth (preopACD) of an anterior chamber of human eyes, a second parameter as the horizontal length (preopAL) of human eyes and a third parameter as the postoperative position of intraocular lenses in human eyes,
wherein specific value triplets with each one value of the three mentioned parameters are selected from these values as main interpolation nodes and the main interpolation nodes are entered into a three-dimensional coordinate system spanned by the three parameters, wherein main interpolation nodes are connected by edge connecting lines, and at least by the positions of the edge connecting lines, a predictive network unevenly shaped in the three-dimensional coordinate system for predicting the postoperative horizontal depth (postopeIOLE, postopeIOLE*) of the intraocular lens in the patient's eye is generated, wherein parameters of the patient's eye are measured and provided for the prediction of the depth, and depending on the measured parameters of the patient's eye and on the predictive network, the postoperative horizontal depth (postopeIOLE, postopeIOLE*) is determined and provided.

2. The method according to claim 1, wherein the postoperative horizontal depth (postopeIOLE, postopeIOLE*) of the intraocular lens in the patient's eye to be preoperatively predicted is determined depending on at least one connecting line contributing to the generation of the predictive network.

3. The method according to claim 1, wherein the predictive network is generated from a plurality of partial meshes, and the main interpolation nodes are selected such that they each are vertices of at least one partial mesh, in particular the main interpolation nodes are selected such that the partial meshes are spanned as rectangles upon projection of the edge connecting lines bounding them into the coordinate plane, which is spanned by the mutually perpendicular coordinate axes for the parameter of the depth (preopACD) of the anterior chamber and the parameter of the length (preopAL) of the human eye, and constitute quadrants of the overall area, in particular the rectangles are introduced into the coordinate system such that the edge connecting lines extend parallel to the coordinate axes between each two vertices of a partial mesh upon projection of the edge connecting lines into the coordinate plane, which is spanned by the mutually perpendicular coordinate axes for the parameter of the depth (preopACD) of the anterior chamber and the parameter of the length (preopAL) of the human eye.

4. The method according to claim 1, wherein the main interpolation nodes are selected such that the lengths of the edge connecting lines of the quadrants projected into a coordinate plane spanned by the coordinate axes characterized by the parameters of the depth of the anterior chamber and the length of the eye are differently generated in the direction of the first coordinate axis (preopACD, preopAL) in the coordinate plane and/or the main interpolation nodes are selected such that the lengths of the edge connecting lines of at least two quadrants projected into a coordinate plane spanned by the coordinate axes characterized by the parameters of the depth of the anterior chamber and the length of the eye are differently generated in the direction of the second coordinate axis (preopACD, preopAL) in the coordinate plane.

5. The method according to claim 1, wherein depending on the pair of values of the depth (preopACD) of the anterior chamber of the patient's eye and the length (preopAL) of the patient's eye, that partial mesh with the associated quadrant of the predictive network is selected, by which the pair of values in the coordinate axes (preopACD, preopAL) is encompassed.

6. The method according to claim 5, wherein for the value of the depth (preopACD) of the anterior chamber of the patient's eye, a first intermediate connecting line contributing to the generation of the predictive network is determined depending on the values of the edge connecting lines of the selected partial mesh at the location of the value of the depth (preopACD) of the anterior chamber of the patient's eye, and/or for the value of the length (preopAL) of the patient's eye, a second intermediate connecting line contributing to the generation of the predictive network is determined depending on the values of the edge connecting lines of the selected partial mesh at the location of the value of the length (preopAL) of the patient's eye.

7. The method according to claim 6, wherein at the location of the pair of values of the depth (preopACD) of the anterior chamber and the length (preopAL) of the patient's eye, at least one predictive value of the postoperative depth (postopeIOLE, postopeIOLE*) of the intraocular lens in the patient's eye is calculated from at least one intermediate connecting line, in particular each one predictive value is calculated from both intermediate connecting lines, and the two predictive values are compared, and depending on an occurring difference of the predictive values, the further mode of determination of a final predictive value of the postoperative depth (postopACD, postopeIOLE) of the intraocular lens in the patient's eye is determined for preoperative prediction, in particular an average determination is determined.

8. The method according to claim 1, wherein for the determination of the postoperative value of the depth (postopeIOLE, postopeIOLE*) of the intraocular lens in the patient's eye for a provided intraocular lens with a haptic inclined with respect to the vertical and/or for a specific provided implantation location of the intraocular lens in the patient's eye, a first correction factor is taken into account, in particular a correction factor is taken into account, which is variably determined depending on the lens shape and/or the implantation location in the patient's eye.

9. The method according to claim 8, wherein the correction factor (Fakₖₒᵣᵣ) is determined by the following functional correlation:
Fakₖₒᵣᵣ−−a+[(Fak_{Z}·b)−c];
wherein a is between 1.3 and 1.43, b is between 0.45 and 0.65 and c is between 60 and 70, preferably between 64 and 66,
in particular, it applies:
Fakₖₒᵣᵣ=−1.39329+[(Fak_{Z}·0.5663)−65.60],
and in particular the factor FakZ is between 110 and 125 and/or the factor FakZ is determined as a function, in particular a linear function, depending on a parameter of the intraocular lens, in particular the refractive power thereof.

10. The method according to claim 1, wherein the values of the depths of anterior chambers of human eyes provided for selecting the main interpolation nodes are determined by measuring human eyes and/or values of lengths of human eyes are determined by measuring human eyes and/or values of postoperative depths of intraocular lenses in human eyes are measured and/or estimated and/or calculated, in particular measured values are only taken into account at main interpolation nodes, which are connected to edge connecting lines circumferentially bounding the predictive network.

11. The method according to claim 1, wherein the main interpolation nodes are preset in user-defined manner.

12. The method according to claim 1, characterized in that wherein the predictive network and/or the determined and preoperatively predicted postoperative horizontal depth (postopeIOLE, postopeIOLE*) of the intraocular lens is displayed on a display unit of a device, in particular the determined and preoperatively predicted postoperative horizontal depth (postopeIOLE, postopeIOLE*) of the intraocular lens is displayed on a display unit of a device as a value and/or in an eye presented as an image.

13. The method according to claim 1, wherein depending on the preoperatively predicted postoperative horizontal depth (postopeIOLE, postopeIOLE*) of the intraocular lens in the patient's eye, the intraocular lens is modeled in its shape and/or refractive power.

14. The method according to claim 1, wherein a once generated predictive network is corrected depending on subsequently obtained information about the actual positional depth of the intraocular lens in the patient's eye after implantation thereof and in particular corresponding main interpolation nodes are in particular automatically changed.

15. The device for performing a method according to claim 1, which has at least a means in order to determine the predictive network and includes at least a means to determine the predictive value.
